SEQUENCE LISTING (14 sequences)

SEQ ID NO: 1 - DNA, 1260 bp, synthetic construct
  source 1..1260: mol_type = other DNA
atgcgttttgaagaatgggttaaagataaacatatcccattcaaacttaatcatccagatgataactacgatgattttaaaccacttcgtaaaattatcggtgatacaagagttgttgctttaggtgaaaattctcacttcattaaagagttcttcttattacgtcacacattattacgtttcttcattgaagatcttggttttacaacattcgcttttgaatttggttttgctgaaggtcaaattattaataactggattcatggtcaaggtacagatgatgaaattggtagatttttaaagcacttctattacccagaagagttaaaaacaacatttttatggcttcgtgagtacaataaagctgctaaagaaaaaatcacgtttcttggtattgatattccaagaaatggtggttcttatttaccaaatatggaaattgttcacgacttttttcgtacagctgataaagaagctttacacattattgatgatgctttcaatatcgcgaaaaaaatcgattatttctctacatctcaagctgctcttaatttacatgaattaacagattcggagaaatgtcgtttaacatctcaattagctcgtgttaaagttcgtttagaagctatggctccaattcatattgaaaaatatggtatcgacaagtatgaaacaattcttcattatgctaacggtatgatctatcttgattataacattcaagctatgtctggtttcatttctggtggtggtatgcaaggtgatatgggtgctaaagataaatatatggctgattctgttttatggcaccttaaaaatccacaatctgaacaaaaagtcatcgttgttgctcataatgctcatattcaaaaaacaccaatcctttatgatggttttctttcttgtttaccaatgggtcaacgtttaaaaaatgctattggtgatgattacatgtctttaggtattacatcttattctggtcatacagctgctttatatccagaagttgatacaaaatatggttttcgtgttgataacttccaattacaagaaccaaatgaaggttctgttgaaaaagctatttctggttgtggtgttacaaattctttcgtttttttccgtaacattccagaagatttacaatctattccaaacatgattcgtttcgattctatctatatgaaagctgaacttgaaaaagcttttgatggtattttccaaatcgaaaaatcttctgtttcggaagttgtttatgagtaa

SEQ ID NO: 2 - DNA, 1000 bp, Oocystis sp.
  source 1..1000: mol_type = other DNA
tacgtggtggtttagatttcactaaagatgatgaaaacgtaaactcacaaccttttatgcgttggagagatcgtttcttattcgtagcagaagctacttataaagcacaagcagaaacaggtgaagttaaaggtcactatttaaatgcaactgctggtacttctgaagaaatgatgaaacgcgcagtttgtgcaaaagaactaggtgtaccgatcattatgcacgattacattactgcaggtttcactgcaaatactagtttagcaaattactgtcgtgataacggtctacttcttcatattcaccgtgcaatgcacgcagttattgaccgtcaaagaaaccatggtattcacttccgtgttttagctaaaattcttcgtatgtctggtggtgaccatttacactcaggtacagttgtaggaaaactagaaggtgaacgtgaagtaacactaggtttcgttgatttaatgcgtgatgattacatcgaaaaagatcgtagtcgtggtatttatttcactcaagactgggcttctttacctggtgtaatgccagtagcttctggtggtatccacgtatggcatatgccagctcttgttgaaatctttggtgatgacgcttgtttacagtttggtggtggtacattaggccatccttggggtaatgctccaggtgctgttgcaaaccgtgttgctctagaagcttgtacacaagctcgtaacgagggtcgtgatttagctcgtgaaggtggtgacatcattcgtgcagcttgcagatggagtccagaactagcagcagcttgtgaagtttggaaagaaattaaatttgaattcgatactatcgataaactataattttttctttacaaattcttatctcttgattttttagtaaaaatattttactaatttatacttttcgattgactttttagttaactaagattcaattgaaaaagttcaacaagaaataaattctaaagattcttaaaaaagaatatcac

SEQ ID NO: 3 - DNA, 1001 bp, Oocystis sp.
  source 1..1001: mol_type = other DNA
ttcggttgtcaaaccctgtttgacaaccaaagtgataaaaaaataactaatatatacttttttctattagcatttattagcttttaacccctaaagcactttttagtcatgcaacctatttattaataaaactttatctttaacaaactagcttttacaaagaagttatattttgattctattaatatagtaaataatttccttaggtcaaacaaagtttgactacacacgcagtgtatagaaaaataaaactataaaaatcaaaaaaccttcgaattatttcggaaagtcagattgctattttataaagttatatttactttccaaaaagtaaatacttataaaatttaataaaaagtagataaaataggaagaagtctacaaattaaaaataaaaagagaaaagttaagaaattgtttcacaaagaactttagtaaaggtttttctattcgtaaaaaaaaaagaaatccttttctacgatatctttgaaaaaacaaatcgttgtttttagattttaggagaaattcgatgacaattagcccaccagagcgtgaagcaaaaaaggtgaaaattgtagtcgatcgaaatcccgttgaaacaagttttgaaaaatgggcaaaaccaggtcatttttcaagaacgttagcgaaaggtccaaatactactacatggatttggaatcttcatgcagatgcacatgatttcgatactcaaactagtgatttagaagaaatctctagaaaagtattcagtgcacattttggtcaattaggtattatcttcatttggcttagtggtatgtatttccatggtgcacgtttctctaattatgaagcatggttaagtgatccaacgcatattaaaccaagtgcacaagtagtttggcctattgttgggcaagaaattttaaatggtgatgttggtggtggattccagggaattcaaattacatcaggtttcttccagctttggcgagctagtggtattactagcgaattac

SEQ ID NO: 4 - DNA, 270 bp, Oocystis sp.
  source 1..270: mol_type = other DNA
gtagaaattttttttaagtttaagggttctagtcgaataaggtatacacataccaatcaaagattagtatgtgtaagaccaaaagaaagttcaataaaataaaaaaaggagaggcggactggattgacagaaggaaaccgactaagctattgtattaatgtgacttgaaaattctatctatcttttcaataattttgaaaaaggtaagactggtttttgagtgactttcttgataaaaaacgaaggtttttttaaaagaaaaaaatatag

SEQ ID NO: 5 - DNA, 63 bp, unidentified
  source 1..63: mol_type = other DNA; note = T7 bacteriophage
gggagaccacaacggtttcccactagaaataattttgtttaactttaagaaggagatatacat

SEQ ID NO: 6 - DNA, 75 bp, Oocystis sp.
  source 1..75: mol_type = other DNA
ttttataagtttataattttattaatatttcaatacaaatattaataaaatttataaatcagaaattaaagaaag

SEQ ID NO: 7 - DNA, 1911 bp, synthetic construct
  source 1..1911: mol_type = other DNA
atgccactgagccaagaggagagcaccctgatcgagaggtgcttggcaacccctcccattccgccttctgcccggtctgtaaaaaagttgacatcagtcttcccgttgctaagcctggatacgttccttgccttcatcctttttgcagggcaacagccaccatcaacagcatccccatcagcgaggtgcgtatgtcggacgactgcttaccagcgtagggcagacgtcacggttgaagcggataggtgtcggggagctgatcgctcaaggaacatttgttttggagccttttagatagcactttcgcgtgcgtatttcctgatgcccttctgcacggtgttccgacactgcggtgcacgatcaacggcgcggtggctaggcgatgacctagccgctgcctagcacgcaatcccttgtgacgccaactgttcttcttaaacttttctaactgcaggactacagcgtggcatctgcagcactgagcagcgatggccgcatctttactggcgtgaacgtgtaccacttcacaggtgagatcgcttagacagcgctcccagggggctgggaaaagttgctcgcatttttcacaacattcctgcaactgtagccctccattgcctgatatttactggatctgtgaaatttattgtcttgcaactatgtccctcctactctgaattttcgttgctgacactgcccccttcccccgcttgacccaacgacacaaacgcccaaataggtggcccctgcgcagagctcgtcgtgctgggcacagccgcagcagcagccgcaggtaatgcgctagagttgtccatgcatatgatgtatagcaacggtgggtatatgtggggagggtggggattggaacggggctcgttgtactttcaacatgaagtaaagcctacctcttgtattgcccgtttgcaggtaacttgacctgcatcgtggccatcggcaacgagaaccgcggcattctgagcccatgcggccgctgccgccaggtgtgtttatgctgtgcatggtttgtgtgtgtgcatgcgagcgaatgagtggcactgtcagtccgtgaatttccaaacgacggtttgcccaattggcaaaccttcttgccaaccttttttcctagcttttcatattcgtcttgcccctctcacaactgacattgtttctacaactgaacaccactgccgacgtcgcaccttgtccaggtgctgctggacctgcatcccggcatcaaggcaatcgtgaaggtatgttatttaccccctattaagcaacaacttgcatgttttttgcctttcacttacttgatgggcagtttttgccaaaccgaaggtgtgcaaccacatagcaacggggacagcactgttgttgttgttcctgtccactatgtccctgcctccacctggccatgccactcacccgtactgaaccggaaatgtgcaaaccatgtgcaggacagcgacggccagccaacagcagtaggtgcgttacattttcaatgggagtggggaaatcaagcatagttgcagcctaaaagttgggtgagacgggtggtttgtagttgaagtgcaggtgggtggtagggatggggagggcagttctcatgcttgttttgtttgcttgatttttaagaccttgttatttggagctcttgagccttttattattcgtgcgcatgtgtgtgtgcttgcggtggtggaggcagtcgacatggttttgcaccccaacatcttgtgtgcaggctcttgcatgcttgtgcctgctcacacacgcacatagtataaaacacacacagacatgcgcactcatatgtacacaaacgcatattgctcctgcatatcctgccgcgtctactgctgtgcaggtattagggagctgctgccaagcggctacgtctgggagggctaa

SEQ ID NO: 8 - DNA, 75 bp, Oocystis sp.; identical to SEQ ID NO: 6
  source 1..75: mol_type = other DNA

SEQ ID NO: 9 - DNA, 261 bp, Oocystis sp.
  source 1..261: mol_type = other DNA
tgcaaggcgaacatctttgcgagatgatatctcttttgttttatgggttcgttcttcactagtgtatgcccggcagcccagggcaaatcccacccaatgaaattagcctaatccatgccagggcacagcaactcctcgccgtgagatcgatgggccagcaacccggaacaaggctgccagcttgaggtgataaacgcacactttttctgctctcatacttctatctgcttccatactcttcctcacaccacactcggtaaa

SEQ ID NO: 10 - DNA, 2884 bp, synthetic construct
  source 1..2884: mol_type = other DNA
gcagcgagtcagtgagcgaggaagcggaagagcggccgccagttggtacggcatattatggtttggatcctgcaaggcgaacatctttgcgagatgatatctcttttgttttatgggttcgttcttcactagtgtatgcccggcagcccagggcaaatcccacccaatgaaattagcctaatccatgccagggcacagcaactcctcgccgtgagatcgatgggccagcaacccggaacaaggctgccagcttgaggtgataaacgcacactttttctgctctcatacttctatctgcttccatactcttcctcacaccacactcggtaaaatgccactgagccaagaggagagcaccctgatcgagaggtgcttggcaacccctcccattccgccttctgcccggtctgtaaaaaagttgacatcagtcttcccgttgctaagcctggatacgttccttgccttcatcctttttgcagggcaacagccaccatcaacagcatccccatcagcgaggtgcgtatgtcggacgactgcttaccagcgtagggcagacgtcacggttgaagcggataggtgtcggggagctgatcgctcaaggaacatttgttttggagccttttagatagcactttcgcgtgcgtatttcctgatgcccttctgcacggtgttccgacactgcggtgcacgatcaacggcgcggtggctaggcgatgacctagccgctgcctagcacgcaatcccttgtgacgccaactgttcttcttaaacttttctaactgcaggactacagcgtggcatctgcagcactgagcagcgatggccgcatctttactggcgtgaacgtgtaccacttcacaggtgagatcgcttagacagcgctcccagggggctgggaaaagttgctcgcatttttcacaacattcctgcaactgtagccctccattgcctgatatttactggatctgtgaaatttattgtcttgcaactatgtccctcctactctgaattttcgttgctgacactgcccccttcccccgcttgacccaacgacacaaacgcccaaataggtggcccctgcgcagagctcgtcgtgctgggcacagccgcagcagcagccgcaggtaatgcgctagagttgtccatgcatatgatgtatagcaacggtgggtatatgtggggagggtggggattggaacggggctcgttgtactttcaacatgaagtaaagcctacctcttgtattgcccgtttgcaggtaacttgacctgcatcgtggccatcggcaacgagaaccgcggcattctgagcccatgcggccgctgccgccaggtgtgtttatgctgtgcatggtttgtgtgtgtgcatgcgagcgaatgagtggcactgtcagtccgtgaatttccaaacgacggtttgcccaattggcaaaccttcttgccaaccttttttcctagcttttcatattcgtcttgcccctctcacaactgacattgtttctacaactgaacaccactgccgacgtcgcaccttgtccaggtgctgctggacctgcatcccggcatcaaggcaatcgtgaaggtatgttatttaccccctattaagcaacaacttgcatgttttttgcctttcacttacttgatgggcagtttttgccaaaccgaaggtgtgcaaccacatagcaacggggacagcactgttgttgttgttcctgtccactatgtccctgcctccacctggccatgccactcacccgtactgaaccggaaatgtgcaaaccatgtgcaggacagcgacggccagccaacagcagtaggtgcgttacattttcaatgggagtggggaaatcaagcatagttgcagcctaaaagttgggtgagacgggtggtttgtagttgaagtgcaggtgggtggtagggatggggagggcagttctcatgcttgttttgtttgcttgatttttaagaccttgttatttggagctcttgagccttttattattcgtgcgcatgtgtgtgtgcttgcggtggtggaggcagtcgacatggttttgcaccccaacatcttgtgtgcaggctcttgcatgcttgtgcctgctcacacacgcacatagtataaaacacacacagacatgcgcactcatatgtacacaaacgcatattgctcctgcatatcctgccgcgtctactgctgtgcaggtattagggagctgctgccaagcggctacgtctgggagggctaagcacctaagcacttgcgcgactccctgtaccagcaggatgcgtagtgacctagcttcagcgagtgttgaatggcagggagggtgaacggctgcgtcatcgccgcctcgcccccctgcatttagcatcccgcctgccattttagtgtgaggagagagggaagccccgatgcgaccgtctacttcttaaacgagcacctagcatcggcacctgattgttattatgggggggctgttgcctcccgagctttgctttgagtggagggcgggtgcagccctgcccctgaattccatgggggcatgggtgtcccctgcatcaaattgagtgtagtattgtttccatcttcgagatcattttgacttgaaagatgatgcgtggagtatgaatgtaaccttaataaatatcatgccttacagcatgtcacatgtggtgctaggctcgaaaactcgaacctgaggttcaacagctccacgtgaatggcacaggatagtccttgtcgacgtttataggacggtgaagccccttttatgtcaactgcatgtttcttgtaccaaaggccctggcggcctctacgcacagccacacacccaaacatctatcctccagatcaccagggcgcgccgacgaaagggcctcgtgatacg

SEQ ID NO: 11 - DNA, 23 bp, Oocystis sp.
  source 1..23: mol_type = other DNA
gtttccagcggagataacgaagg

SEQ ID NO: 12 - DNA, 26 bp, Oocystis sp.
  source 1..26: mol_type = other DNA
ctcctccctttcatcccttacagatg

SEQ ID NO: 13 - DNA, 22 bp, Oocystis sp.
  source 1..22: mol_type = other DNA
gaacttgctcatcacctccttg

SEQ ID NO: 14 - DNA, 23 bp, Oocystis sp.
  source 1..23: mol_type = other DNA
cttaaaaaagaatatcacttcgg

(19) United States
(12) Patent Application Publication
(10) Pub. No.: US 2025/0043296 A1
(43) Pub. Date: Feb. 6, 2025

(54) METHOD FOR NUCLEAR GENOME EDITING USING PLASTID SELECTABLE MARKERS

(71) Applicant: VIRIDOS, INC., La Jolla, CA (US)

(72) Inventors: John Verruto, La Jolla, CA (US); Daniel Strimling, La Jolla, CA (US)

(21) Appl. No.: 18/793,396

(22) Filed: Aug. 2, 2024

Related U.S. Application Data
(60) Provisional application No. 63/530,816, filed on Aug. 4, 2023.

(51) Int. Cl. C12N 15/82; C12N 9/22; C12N 15/11; C12N 15/89

(52) U.S. Cl. CPC C12N 15/8209; C12N 9/22; C12N 15/111; C12N 15/895; C12N 2310/20

(57) ABSTRACT

The invention provides methods of using chloroplast-selectable markers to detect nuclear genome editing. The methods involve selecting a genetic modification that has occurred in a nuclear genome of a photosynthetic cell; transforming a photosynthetic cell with a ribonucleoprotein complex that effects a genetic modification in the genome in the nucleus of a photosynthetic cell; transforming the photosynthetic cell with a construct that effects an insertion of a selectable marker into the plastome of a plastid of the cell; selecting for photosynthetic cells having the selectable marker inserted into the plastome; and thereby selecting for the genetic modification to the genome in the nucleus of the photosynthetic cell.

### CROSS-REFERENCE TO RELATED APPLICATIONS

This application claims benefit of priority under 35 U.S.C. § 119(e) of U.S. Provisional Application No. 63/530,816, filed Aug. 4, 2023. The disclosure of the prior application is considered part of and is herein incorporated by reference in the disclosure of this application in its entirety.

### INCORPORATION OF SEQUENCE LISTING

The material in the accompanying sequence listing is hereby incorporated by reference into this application. The accompanying sequence listing xml file, name SG12310-1.xml, was created on Jul. 30, 2024, and is 22158 kb.

### FIELD OF THE INVENTION

The invention involves selection methods for detecting organisms having a nuclear genome modification.

### BACKGROUND OF THE INVENTION

Genome editing is a rare event, and thus often requires a means of down-selecting the transformed population prior to screening clones for the editing event. This is usually achieved by co-transformation of a dominant selectable marker and selection for resistance to inhibitory compounds such as antibiotics or herbicides. In many organisms nuclear genome editing has been demonstrated by utilizing co-transformation of nuclear selectable markers. But in organisms where the number of developed nuclear markers are limited, the stacking of edits relies heavily on multiplexing different Cas9-ribonucleoproteins to simultaneously edit multiple targets in a single transformation. This process can be laborious and inefficient and often creates dead end strains once the nuclear markers are spent. Additional disadvantages include that several green algae have only a few nuclear selectable markers that can be utilized, due either to the organism's insensitivity to inhibitory compounds, or challenges inherent in expressing the nuclear marker transgene (e.g. strong epigenetic silencing).

While the recycling of nuclear markers is achievable in certain organisms using site specific recombinases this approach requires the ability to reliably express additional transgenes and/or to select for the delivery of the recombinant Cre protein, which then runs into the same initial problem of selecting for a rare event. This nuclear marker recycling approach is further restricted in organisms where aggressive non-homologous end joining and concatemer formation of selectable marker cassettes often result in strains harboring high copy numbers of the nuclear marker, and often as tandem repeats in varying orientations. Strains exhibiting this type of integration leave no way of removing markers, even if Cre expression or delivery were reliable.

It would therefore be useful to have markers that are easy to integrate into the organism and easy to express, and that do not require expensive instrumentation to use or extensive method development.

### SUMMARY OF THE INVENTION

The invention provides methods of using plastid-selectable markers to detect nuclear genome editing. The methods involve selecting for a genetic modification that has occurred in a nuclear genome of an algal cell. The method involves transforming an photosynthetic cell with a ribonucleoprotein complex that effects a genetic modification in the genome in the nucleus of an algal cell, transforming the photosynthetic cell with a construct that effects an insertion of a selectable marker into the plastid or plastome of the cell, selecting for cells having the selectable marker inserted into the plastid or plastome, and thereby selecting for the genetic modification to the genome in the nucleus of the photosynthetic cell.

In a first aspect the invention provides methods of selecting for a genetic modification to a genome in the nucleus of a photosynthetic cell. The methods involve transforming the photosynthetic cell with a ribonucleoprotein complex that effects a genetic modification in the genome in the nucleus of the photosynthetic cell; transforming the photosynthetic cell with a construct that effects an insertion of a selectable marker into the plastid of the photosynthetic cell; selecting for photosynthetic cells comprising the selectable marker inserted into the plastid; and thereby selecting for the genetic modification to the genome in the nucleus of the photosynthetic cell. In one embodiment the insertion of the selectable marker into the plastid of the cell comprises inserting the selectable marker into the plastome. The methods can also involve sequencing at least a portion of the genome in the nucleus of the photosynthetic cell to confirm the genetic modification has been performed. The method of claim 2 wherein selecting for photosynthetic cells comprises plating algal cells and selecting colonies comprising the insertion into the plastome.

In some embodiments the selectable marker is inserted into the plastome and enables the photosynthetic cell to grow on an antibiotic. In various embodiments the antibiotic can be erythromycin or spectinomycin. In some embodiments the selectable marker can be glufosinate ammonium or the ptxD gene. In any embodiment the cells can be plated onto selection agar plates. The ribonucleoprotein complex can contain a guide RNA targeted to a sequence on the genome in the nucleus of the photosynthetic cell, and a Cas nuclease. In any embodiment the method can not include (or can omit) a step of screening for a selectable marker present in the genome of the nucleus of the photosynthetic cell.

In any embodiment the photosynthetic cell can be an algal cell, for example a Trebouxiophyte algal organism, for example from the genus Oocystis. The construct that effects an insertion of a selectable marker into the plastid can have RS-up and RS-down arms that recombine with homologous sequences in the plastome, and thereby effect the insertion. The construct can also have a selectable marker cassette in between the RS-up and RS-down arms. In one embodiment the construct is amplified by PCR prior to being transformed into the plastid. In one embodiment the construct can have a chloroplast promoter and chloroplast terminator that controls expression of the encoded selectable marker. In any embodiment the encoded selectable marker can confer the ability to grow on media containing erythromycin, spectinomycin, or glufosinate ammonium. In one embodiment the encoded selectable marker is the ptxD gene. In any embodiment the transformations can be performed using biolistics.

In another aspect the invention provides compositions containing a ribonucleoprotein complex for editing a nuclear genome, and a construct having a selectable marker comprised in between two restriction site arms, each restriction site arm being homologous to corresponding sequences on a plastid genome. The composition of claim 19 wherein the ribonucleoprotein complex comprises a guide RNA targeted to a site on the nuclear genome of a subject organism. In one embodiment the ribonucleoprotein complex comprises Cas9. In one embodiment the subject organism is a Chlorophyte organism, for example a Trebouxiophyte alga. In one embodiment the composition can be adsorbed to a microcarrier for biolistics (e.g. a microbead).

In another aspect the invention provides kits containing any of the compositions described herein.

### DETAILED DESCRIPTION OF THE DRAWINGS

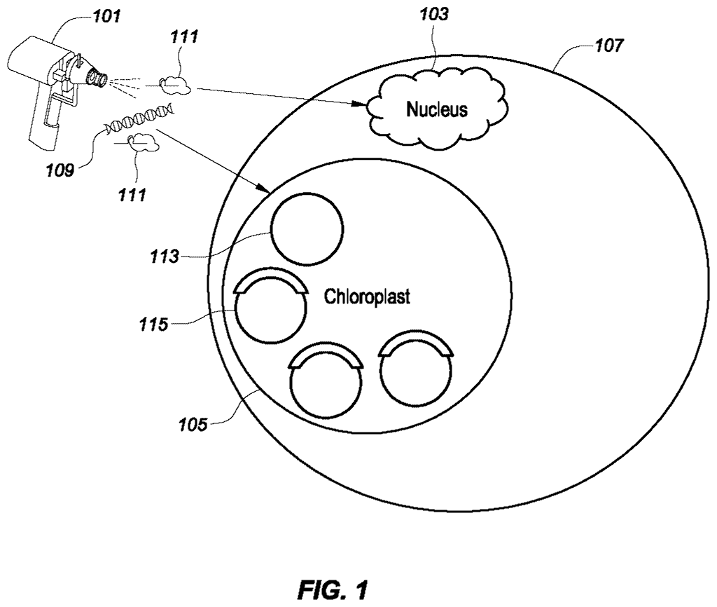

FIG. 1 is a schematic illustration of an embodiment of the invention involving nuclear editing with chloroplast selection markers. Depicted are a gene gun 101 utilized for transformation into the nucleus 103 and chloroplast 105 of the cell 107. Also depicted are chloroplast marker 109 with homology arms and Cas9-RNP 111, which can be directed to both the nucleus and chloroplast. Also depicted are the wild-type plastome 113 and the transgenic plastome 115 with selectable marker.

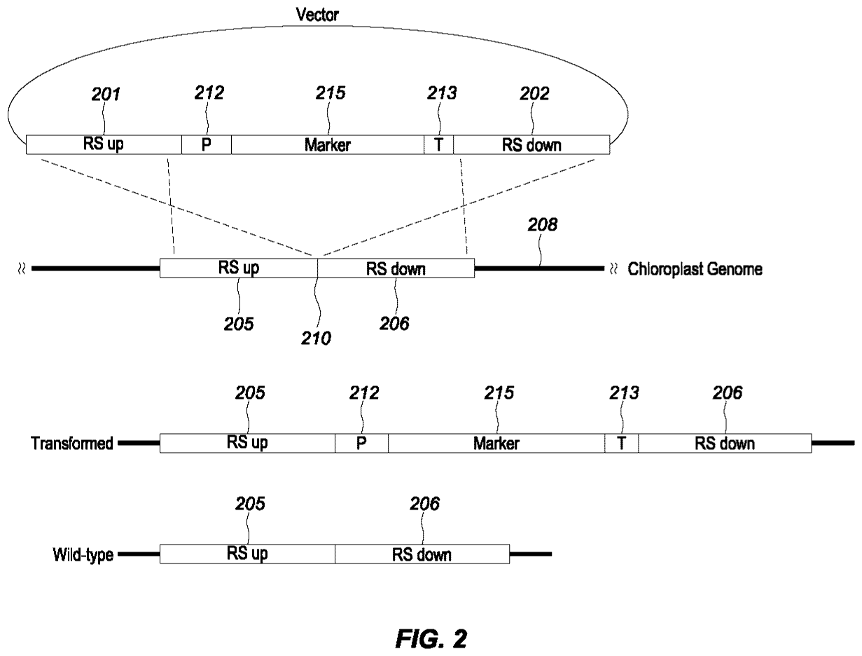

FIGS. 2A-2B; FIG. 2A provides a schematic illustration of transformation vector design and integration of a selectable marker into the chloroplast genome. Depicted is the vector 203 with RS-up arm 201 and RS-down arm 202, and selectable marker 215 with homologous sequences 205 and 206 in the chloroplast genome. FIG. 2B illustrates the resulting transformed and original wild-type locus.

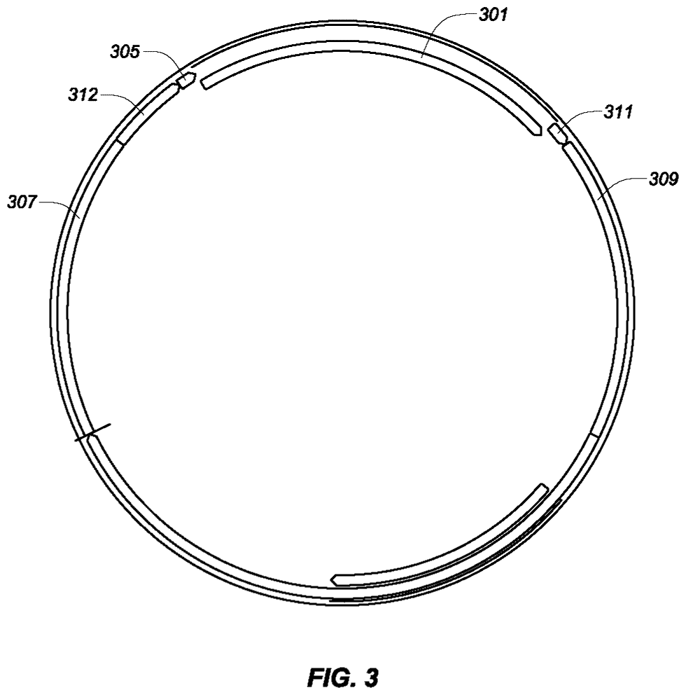

FIG. 3 provides a vector map for the chloroplast integration vector NAS30505. The vector has 5,692 bp. Depicted are the RS3 up arm 307 and RS3 down arm 309, a viral T7g10 5′ UTR 305 and an atpB terminator 311. A selectable marker ereB 301 is also depicted. The vector is based on a pUK minimal vector backbone.

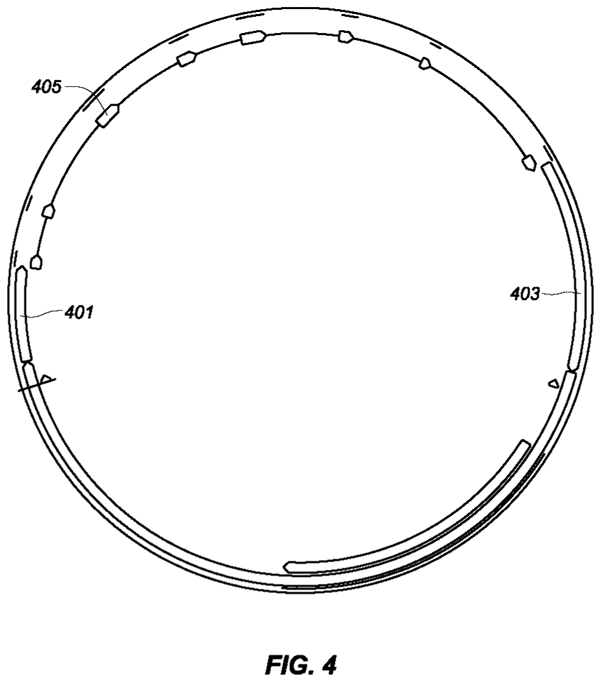

FIG. 4 provides a vector map for the nuclear transformation vector NAS16305. The vector has 4,788 bp. Depicted are an ACP promoter 401 and ACP terminator 403. Also depicted is the BSD selectable marker 405 with endogenous introns. The vector is also based on the pUK minimal vector backbone.

### DETAILED DESCRIPTION OF THE INVENTION

The invention provides methods and compositions for selecting for a genetic modification in the nuclear genome of a photosynthetic cell. The methods involve utilizing a plastid (e.g. chloroplast) selectable marker that, when transformed into the plastid or plastome, correlates with the occurrence of the genetic modification in the nuclear genome. The methods and compositions therefore enable users to avoid the problem of running out of nuclear markers to achieve nuclear genome editing. The invention provides many advantages over the use of nuclear markers. For example, compared to nuclear markers plastid (e.g. chloroplast) markers are easier to express as they are free of epigenetic silencing that is often a problem with expressing nuclear markers. The plastid genome is also easy to integrate into using homologous recombination. The invention also provides the ability to replace markers and/or swap out markers in the plastid in subsequent steps when recycling of markers is desirable. The invention therefore allows for robust marker recycling strategies and negative selection markers are also available. The present inventors discovered unexpectedly that plastid selectable markers can be utilized to efficiently select for modifications that have occurred in the nuclear genome.

The methods involve selecting for a genetic modification that has occurred in the nucleus of a photosynthetic cell. The methods can involve a step of transforming a photosynthetic cell with a ribonucleoprotein complex (RNP) that effects a genetic modification in the genome in the nucleus of the photosynthetic cell. The invention also involves transforming a photosynthetic cell with a construct that effects an insertion of a selectable marker into the plastid or plastome of the cell, selecting for cells that have the selectable marker inserted into the plastid or plastome, and thereby selecting for the genetic modification to the genome in the nucleus of the cell. The selecting can also involve plating or cultivating the photosynthetic cells and identifying colonies or samples that have the insertion into the plastid or plastome. For example, if the selectable marker is ereB then growth on a medium containing erythromycin indicates the growing photosynthetic cells have picked up the ereB gene. Plating can refer to growth of the organisms on a solid or semi-solid medium (e.g. agar), but can also refer to growth in liquid medium. But in other embodiments the cells could be cultivated in liquid or semi-solid medium and selected by any means indicating growth in the medium. Plastids are cellular organelles that contain their own genomes, termed plastomes. In various embodiments plastids can be chloroplasts, chromoplasts, or leucoplasts. Plastids often contain pigments used in photosynthesis. In any embodiment the plastid can be a chloroplast. In any embodiment the photosynthetic cell can be an algal cell, e.g. a green alga or a Chlorophyte alga. The plastome can be a double-helix-circular DNA molecule. In various embodiments the plastome can be 120-160 kb in size.

In one embodiment the construct is transformed into the plastid of the cell, but does not necessarily insert into the plastome. For example, the construct can be carried as one or more episomal DNA molecules inside the plastid. In some embodiments these episomes can be replicated autonomously in the photosynthetic cell. In any embodiment the episomes can be free, circular, extrachromosomal DNA molecules.

### Genetic Modifications

A “genetic modification” applied in the invention can be any genetic modification. In some embodiments the genetic modification is one that can be performed by a ribonucleoprotein complex or by homologous recombination. In various embodiments the genetic modification can be one or more of an attenuation, a deletion, a gene “knock out,” one or more mutations or sequence changes, a disruption, an insertion, insertion of a stop codon, an inactivation, a rearrangement, one or more point mutations, a frameshift mutation, a nonsense mutation, an inversion, a single nucleotide polymorphism (SNP), a truncation, a point mutation, or any combination of the above modifications that changes the activity or expression of the one or more gene or nucleic acids. In any embodiment the insertion can be an insertion into the genome and/or the plastid or plastome of the cell. In some embodiments the change in expression is a reduction in expression or an elimination of the expression or activity, which expression can be the production of an encoded protein. The genetic modification can be made or be present in any sequence that affects expression or activity of a gene or nucleic acid sequence, or the nature or quantity of its product, for example to a coding or non-coding sequence, a promoter, a terminator, an exon, an intron, a 3′ or 5′ UTR, or other regulatory sequence. A genetic modification can be performed in any structure of the gene, and in some embodiments results in attenuation or elimination of the gene or nucleic acid product or activity. In various embodiments the genetic modification is a deletion, disruption, or inactivation. The genetic modification can be made to, or be present in, the host cell's native nuclear genome or in the plastid or plastome. In some embodiments, a photosynthetic cell or organism having attenuated expression of a gene can have one or more mutations, which can be one or more nucleobase changes and/or one or more nucleobase deletions and/or one or more nucleobase insertions, into the region of a gene 5′ of the transcriptional start site, such as, in non-limiting examples, within about 2 kb, within about 1.5 kb, within about 1 kb, or within about 0.5 kb of the known or putative transcriptional start site, or within about 3 kb, within about 2.5 kb, within about 2 kb, within about 1.5 kb, within about 1 kb, or within about 0.5 kb of the translational start site.

An “attenuation” is a genetic modification resulting in a reduction of the function, activity, or expression of a gene or nucleic acid sequence compared to a corresponding (control) cell or organism not having the genetic modification being examined, i.e. the diminished function, activity, or expression is due to the genetic modification. The activity of a nucleic acid sequence can be expression of an encoded polypeptide, a binding activity, the amount of signal transduction or transcription regulation, or other activity the nucleic acid sequence exerts within the organism. In various embodiments an attenuated gene or nucleic acid sequence disclosed herein produces less than 90%, or less than 80%, or less than 70%, or less than 50%, or less than 30%, or less than 20%, or less than 10%, or less than 5% or less than 1% of its function, activity, or expression of the gene or nucleic acid sequence compared to the corresponding (control) cell or organism. In various embodiments a gene attenuation can be achieved via a deletion, a disruption, or an inactivation. Any of the genetic modifications described herein can result in partial or complete attenuation of the function, activity, or expression of the attenuated gene or nucleic acid sequence, which in some embodiments can lead to a level of function, activity, or expression that is not substantially more than that of complete attenuation; for example, the function, activity, or expression can yield a result that is less than 10% different from a recombinant cell or organism having a complete attenuation of the gene or nucleic acid sequence.

In any embodiment the genetic modification can be intentionally induced by human activity, or “man-made” genetic modification. Thus, in any embodiment the genetic modification can be a modification that was not introduced by continuous culturing or ambient uv light (or similar or shorter wavelength light). An unmodified gene or nucleic acid sequence present naturally in the organism denotes a natural, endogenous, or wild type sequence. A deletion can mean that at least part of the object nucleic acid sequence is deleted or that at least part of the encoded product is eliminated or truncated. But a deletion can also be accomplished by disrupting a gene through, for example, the insertion of a sequence into the gene that is not naturally present (e.g. a selection marker), a combination of deletion and insertion, or mutagenesis resulting in insertion of a stop codon. But a deletion can also be performed by other genetic modifications known to those of ordinary skill that result in the loss of expression, activity, or function of a gene or nucleic acid sequence.

Transformation involves the genetic alteration of a cell resulting from the uptake and incorporation of exogenous genetic material. In any embodiment transformation can involve the incorporation of the exogenous genetic material into the nucleus or the plastid or plastome of the cell, e.g. into the nuclear or plastid genome or into the nuclear or plastid space. The transformation can also involve the modification of the nuclear genome and/or plastome, and the ability to activate incorporated genetic material or modifications to express a phenotype. Transformation can involve the genetic modification of native sequences.

Transformation of the cells or organisms can be performed by any suitable means. Many methods of transformation are known to the person of ordinary skill in the art and can be applied in the invention, including but not limited to electroporation, viral transformation, micro-injection, and other methods. In some embodiments transformation can be conveniently accomplished with the use of biolistics (or particle bombardment), which can involve the use of a “gene gun.” Biolistic methods allow for the direct introduction of DNA or RNA into cells. The DNA, RNA, RNP complex, or other material to be transformed can be coated onto particles (e.g. microparticles of gold, tungsten, or another heavy metal) and released from the gene gun under high pressure (e.g. helium gas) to bombard and directly penetrate the cell wall. Many biolistic methods are published and the procedures are known to those of ordinary skill in the art. Biolistics offers the advantage of being able to co-transform multiple genome editing materials into the cell, as well as the ability to deliver large DNA fragments or particles. While biolistic methods may be effective and convenient, and allow for the transformation of organelles including plastids and mitochondria, any method of transformation can be utilized in the methods.

In any embodiment the methods of the invention can produce a transformed or recombinant photosynthetic organism. When applied to organisms, “transformation,” “transgenic” “transformed” or “recombinant” or “engineered” or “genetically engineered” refer to organisms that have been manipulated by introduction of an exogenous or recombinant nucleic acid sequence into the organism, or by genetic modification of native sequences (which are therefore then recombinant). In various embodiments recombinant or genetically engineered organisms can be organisms into which constructs for gene “knock down,” insertion, deletion, attenuation, inactivation, or disruption have been introduced to perform the indicated manipulation. Such constructs include, but are not limited to, DNA constructs, RNAi, microRNA, shRNA, antisense, and ribozyme constructs. A recombinant organism can also include those having an introduced exogenous regulatory sequence operably linked to an endogenous gene of the transgenic microorganism, which can enable transcription in the organism. Also included are organisms whose genomes have been altered by the activity of meganucleases or zinc finger nucleases. A heterologous or recombinant nucleic acid molecule can be integrated into a genetically engineered/recombinant organism's genome or plastome, or, in other instances, can be not integrated into a recombinant/genetically engineered organism's genome or plastome but be present on a vector, episome, or other nucleic acid construct. As used herein, “recombinant microorganism” or “recombinant host cell” includes progeny or derivatives of the recombinant microorganisms of the disclosure.

In any embodiment the genetic modifications performed in the methods can involve human activity, for example, by classical mutagenesis or genetic engineering, but can also involve the use of any feasible mutagenesis method, including but not limited to exposure to UV light (i.e. deliberate exposure and not ambient uv light), CRISPR/Cas9, cre/lox, gamma irradiation (not ambient), or chemical mutagenesis. Screening methods can be used to identify mutants having desirable characteristics (e.g., reduced chlorophyll and increased lipid and/or biomass productivity). The methods can involve performing the genetic modifications of the invention using classical mutagenesis, genetic engineering, and phenotype or genotype screening are known in the art.

### Constructs

The methods involve transforming a photosynthetic cell with a ribonucleoprotein complex that effects a genetic modification described herein in the genome of the algal cell. The methods also involve transforming the photosynthetic cell with a construct that effects an insertion of a selectable marker into the plastid or plastome of the photosynthetic cell. By “effecting” the genetic modification or the insertion is meant to cause and enable it to come about as a result of specific action, e.g. a step of transformation. Thus, a ribonucleoprotein (RNP) complex can have the guide RNA and the Cas protein (e.g. Cas9, other Cas protein, or similar). When transformed into a photosynthetic cell the RNP complex performs the stated genetic modification (e.g. to the nuclear genome). In any embodiment the insertion can comprise insertion of the construct so that the promoter and terminator govern initiation and termination of transcription of the selectable marker gene also comprised on the construct. The “construct” refers to a DNA molecule containing one or more components able to be transformed into the plastid or plastome. In one embodiment the construct can effect insertion of the construct into the plastome. But in another embodiment the construct can function as an episome in the plastid. Examples of transformation methods include, but are not limited to, biolistics, electroporation, liposome-mediated transformation, bacterial conjugation, chemical competency-mediated transformation (e.g. chemically competent E. coli), or any method of transformation known to the person of ordinary skill.

In one embodiment the construct has a vector backbone, an RS-up and RS-down arms, a selectable marker gene, and a promoter and terminator, which may be present on either side of the selectable marker gene to control initiation and termination of transcription of the selectable marker gene. In another embodiment the construct has an RS-up and RS-down arms, a selectable marker gene, and a promoter and terminator, which may be present on either side of the selectable marker gene to control initiation and termination of transcription of the selectable marker gene. While the construct can contain a vector backbone (e.g. from which it was derived) it is not required, and in one embodiment the construct can lack a vector backbone. A DNA construct can be a single-stranded or double-stranded DNA molecule, and can be either linear or circular.

### Photosynthetic Organisms

In any embodiment the photosynthetic cells or organisms (e.g. the subject organism) utilized in the invention can be a microalga, or a photosynthetic organism, or a green alga. In any embodiment the alga can be any eukaryotic microalga such as, but not limited to, a Chlorophyte, an Ochrophyte, or a Charophyte alga. In some embodiments the photosynthetic cell or organism can be a Chlorophyte alga of the taxonomic Class Chlorophyceace, or of the Class Chlorodendrophyceae, or the Class Prasinophyceace, or the Class Trebouxiophyceae, or the Class Eustigmatophyceae. In some embodiments, the algal cell or organism can be a member of the Class Chlorophyceace, such as a species of any one or more of the genera Asteromonas, Ankistrodesmus, Carteria, Chlamydomonas, Chlorococcum, Chlorogonium, Chrysosphaera, Dunaliella, Haematococcus, Monoraphidium, Neochloris, Oedogonium, Pelagomonas, Pleurococcus, Pyrobotrys, Scenedesmus, or Volvox. In other embodiments the algal cell or organism utilized in the invention can be a member of the Order Chlorodendrales, or Chlorellales. In other embodiments, the algal cell or organism can be a member of the Class Chlorodendrophyceae, such as a species of any one or more of the genera Prasinocladus, Scherffelia, or Tetraselmis. In further alternative embodiments, the algal cell or organism can be a member of the Class Prasinophyceace, optionally a species of any one or more of the genera Ostreococcus or Micromonas. Further alternatively, the algal cell or organism can be a member of the Class Trebouxiophyceae, and optionally of the Order Chlorellales, and optionally a genera selected from any one or more of Botryococcus, Chlorella, Auxenochlorella, Heveochlorella, Marinichlorella, Oocystis, Parachlorella, Pseudochlorella, Tetrachlorella, Eremosphaera, Franceia, Micractinium, Nannochloris, Picochlorum, Prototheca, Stichococcus, or Viridiella, or any of all possible combinations or sub-combination of the genera. In another embodiment the algal cell or organism can be a Chlorophyte alga of the Class Trebouxiophyceae and the family Coccomyxaceae, and the genus Coccomyxa (e.g. Coccomyxa subellipsoidea). Or of the family Chlamydomonadaceae and the genus Chlamydomonas (e.g. Chlamydomonas reinhardtii); or of the family Volvocaceae and the genus Volvox (e.g. Volvox carteri, Volvox aureus, Volvox globator).

In another embodiment the photosynthetic cell or organism is a Chlorophyte alga of the Class Trebouxiophyceae, or Eustigmatophyceae, and can be of the Order Chlorellales or Chlorodendrales, and can be of the Family Oocystaceae, or Chlorellaceae, or Monodopsidaceae, and optionally from a genus selected from any one or more of Oocystis, Parachlorella, Picochlorum, Nannochloropsis, and Tetraselmis, in all possible combinations and sub-combinations. The photosynthetic cell or organism can also be from the genus Oocystis, or the genus Parachlorella, or the genus Picochlorum, or the genus Tetraselmis, or from any of all possible combinations and sub-combinations of the genera. In one embodiment the photosynthetic cell or organism is of the Class Trebouxiophyceae, of the Order Chlorellales, and optionally of the family Oocystaceae, and optionally can be of the genus Oocystis.

### Ribonucleoprotein Complex

In any embodiment the ribonucleoprotein (RNP) complex utilized in the invention can be an assembly of a Cas endonuclease and a guide RNA. In various embodiments the Cas endonuclease can be Cas9, Cas12a, Cpf1, or any suitable endonuclease. In one embodiment the ribonucleoprotein complex can be delivered in the methods as a pre-assembled RNP complex, but in other embodiments it can also be delivered as a DNA or RNA construct encoding the genetic instructions for the protein and RNA components. In any embodiment the ribonucleoprotein complex can deliver the CRISPR/Cas9 or Cas9/gRNA system. In some embodiments the ribonucleoprotein complex can effect the genetic modification in the genome in the nucleus of the photosynthetic cell and/or the plastome. The genetic modification can be any described herein.

### Selectable Marker

The invention involves the use of plastid selectable markers. Plastid selectable markers provide selection based on transformation of the selectable marker into the plastid or plastome of an organism. In any embodiment the plastid can be a chloroplast, and the marker is a chloroplast selectable marker. Selection of transformed lines where all copies of the plastid genome have been transformed requires efficient markers. In the invention any suitable trait can be used for selection. In various embodiments selection can be made for photoautotrophy, resistance to antibiotics, or tolerance to herbicides or metabolic inhibitors. In various embodiments the selectable marker can confer the ability to grow on a selection media. In any embodiment the methods can involve a step of plating or otherwise cultivating the photosynthetic cells (e.g. in liquid, solid, or semi-solid medium) where growth on the medium indicates that the selectable marker was inserted into the plastid or plastome of the organism.

Any of the methods can involve a step of selecting for photosynthetic cells or organisms comprising a selectable marker inserted into the plastid or plastome. The selection step can be performed by cultivating the cell or organism on a selection media that permits the growth of organisms that have incorporated the selectable marker into the plastid or plastome. In various embodiments the selectable marker can be transformed into the plastid and/or inserted into the plastome of the cell.

In various embodiments the selection media can contain a concentration of an antibiotic or other agent that prohibits the growth of organisms that have not incorporated the selectable marker, e.g. into the plastid or plastome of the photosynthetic cell. In some embodiments the growth media can have a source of an essential nutrient that organisms can utilize only if they have incorporated the selectable marker, thus permitting the organisms to grow on the media. When the selection agent is an antibiotic it can be erythromycin, spectinomycin, or another suitable antibiotic. The marker can also be the aphA-6 gene, which confers resistance to kanamycin and related inhibitors of 70S ribosomes; the tetX gene, which confers resistance to tetracycline; the arr-2 that confers resistance to rifampicin, and the cat gene, which confers resistance to chloramphenicol. Other antibiotics used for selection are known to persons of ordinary skill. In other embodiments the selection agent can be a gene that confers survival ability to the organism on a medium containing e.g. glufosinate ammonium (e.g. BASTA®, Bayer CropScience, Whippany, NJ), or a gene that confers a survival ability to the organism on a nutrient deficient medium, e.g. the ptxD (phosphonate dehydrogenase) gene that allows organisms to metabolize phosphite in media where it is the only source of phosphorus. The selection marker can also be another suitable gene or marker, or any marker that allows for selection. Non-limiting examples of additional genes that can be used to select transformed organisms include nptII, hpt, bar, and gox, that confer resistance to kanamycin, hygromycin, phosphinothricin, and glyphosate, respectively. In some embodiments selection can be performed on agar plates. In one embodiment the agar plates can have a source of phosphorus that can be accessed only by organisms that have acquired the ptxD gene.

Any embodiment of any of the methods can not include (or can omit or exclude) a step of selecting or screening for a selectable marker present in the genome of the nucleus of the photosynthetic cell. In such embodiments no step is carried out to select for a selectable marker present in the genome of the nucleus of the cell. In any embodiment this step is unnecessary because the screening is advantageously directed to the transformed selectable marker in the plastid or plastome. In some embodiments the methods can involve a step of selecting or screening for multiple selectable marker(s) present in the genome of the cell, for example one, or two, or three selectable markers in the genome can be screened for; but such methods can also involve selecting for at least one selectable marker present in the plastid or plastome of the cell, or two, or three, or more than three selectable markers present in the plastid or plastome of the cell.

In any embodiment a construct carrying the chloroplast selectable marker can effect an insertion into the plastome of the algal cell. In one embodiment the construct is a DNA construct, e.g. a vector. In any embodiment the construct can have RS-up and RS-down arms that are homologous to and recombine with sequences in the plastome to thereby effect insertion of the construct. The construct can encode a selectable marker cassette in between the RS-up and RS-down arms. The RS-up and RS-down arms can be homologous to regions on the plastome of the plastid, and therefore support homologous recombination and insertion of the selectable marker sequence into the plastome. While insertion and/or recombination is conveniently done by homologous recombination, any method of insertion can be utilized.

### Recycling of Markers

As mentioned above, this invention solves the problem of irreversibly “using up” nuclear markers to achieve nuclear editing. Since photosynthetic cells have homologous recombination and segregation pathways acting on chloroplast genomes efficient removal of markers is possible and allows for marker recycling. In various embodiments methods for removing plastid markers can be based on, for example, direct repeats, transient co-integration, or co-transformation and segregation of trait and marker genes. In some embodiments chloroplast markers can be swapped and recycled by homologous recombination in subsequent steps. Robust marker recycling strategies and negative selection markers have been demonstrated. This approach allows for infinite iterative rounds of nuclear editing. (Jackson et al. (2022), Biotechnology Journal, Vol 17, Issue 10, 1-10).

Another advantage of the invention is the ability to restore the chloroplast plastome to its wild-type state, indistinguishable from the native plastome, in nuclear edited lines and leave no residual transformed DNA behind. The only modifications present in the organism after the removal of markers inserted into the plastome are small insertions/deletions (indels) generated by the Cas9-RNP double stranded break and mis-repair by the organisms' NHEJ response. Since no transformed DNA is left behind, the modified organisms can be categorized as non-GMO. The invention therefore can significantly reduce regulatory compliance issues that involve the use of genetically modified organisms outdoors.

### Compositions

The invention also provides compositions comprising a ribonucleoprotein complex for editing a nuclear genome and a construct having a selectable marker comprised in between two restriction site arms homologous to corresponding sequences in a plastid genome. In any embodiment the ribonucleotide complex can contain a Cas protein (e.g. Cas9 or other Cas protein) and a guide RNA. The guide RNA can be complementary to a sequence in an algal nuclear genome of an organism described herein and, with the Cas protein, effect a genetic modification in the algal nuclear genome. The construct can be any described herein, and can have any selectable marker gene described herein. The homology to the corresponding sequences in the plastid genome of a subject organism can enable homologous recombination and insertion of the construct. The construct can be any described herein. In any embodiment the ribonucleoprotein complex and construct can be adsorbed to or present (or co-precipitated onto) on a microcarrier or microparticle suitable for biolistics, for example of gold, tungsten, or another heavy metal.

The invention also provides kits comprising any of the compositions described herein, any combination thereof, and in all possible combinations and sub-combinations. In one embodiment the kit comprises a ribonucleoprotein complex for editing a nuclear genome, and a construct comprising a selectable marker gene comprised in between two restriction site arms homologous to corresponding sequences on a plastid genome of a subject organism (e.g. a photosynthetic organism or cell). In one embodiment the ribonucleoprotein complex and construct are adsorbed or co-precipitated onto a microparticle for biolistics.

### EXAMPLES

### Example 1

This example illustrates the general strategy of vector construction. Constructs for integrating a selectable marker into the plastome of a photosynthetic organism were designed using the following general strategy. A region in the chloroplast genome was selected as the recombination site (RS) 210 for transgene/selectable marker insertion and integration into the plastome. Chloroplast integration vectors were designed to contain homologous RS-up 201 and RS-down 202 arms (FIG. 2A), which recombine with respective homologous sequences 205 and 206 in the chloroplast genome 208 when the DNA is introduced into the organelle (FIG. 2A) The recombination reaction is illustrated as crossed dotted lines. In one embodiment the vector is a DNA construct. To build the vector a selectable marker cassette 215 was inserted in between the RS-up 201 and RS-down 202 arms in the construct. Transgene expression was controlled by a chloroplast promoter 212 and terminator 213 (shown generally in FIGS. 2A-2B) regulating the selectable marker cassette 215. This general approach was followed to build the chloroplast integration vector NAS30505 depicted in FIG. 3.

### Example 2

This example illustrates the use of a chloroplast selectable marker to select for nuclear edits using a chloroplast selectable marker.

To build the chloroplast integration vector NAS30505 depicted in FIG. 3, one RS site 210 referred to as RS3 was chosen for integration, being a large intergenic space between two hairpins. The homologous RS3-up arm 307 (SEQ ID NO 2) and RS3-down arm 309 (SEQ ID NO 3) flank a selectable marker cassette 301 and enable homologous recombination (HR) into the RS3 integration site between the two arm sequences within the plastome 208. The selectable marker cassette includes the erythromycin resistance transgene ereB 301, which was codon-optimized for chloroplast expression as SEQ ID NO 1. The expression of the ereB gene is controlled by a 16S rRNA promoter 312 (SEQ ID NO 4), viral T7g10 5′UTR element 305 (SEQ ID NO 5), and atpB terminator 311 (SEQ ID NO 6).

A nuclear transformation vector was used as a control and named NAS16305, depicted in FIG. 4. This vector contained a BSD gene 405 (blasticidin-S deaminase) codon optimized for nuclear expression and with endogenous introns (SEQ ID NO: 7) transplanted across the coding sequence. The BSD gene expression is controlled by the endogenous ACP promoter 401 (SEQ ID NO: 8) and ACP terminator 403 (SEQ ID NO: 9). The vector was amplified by PCR and the resulting PCR product (NAS16305-PCR, SEQ ID NO 10) was treated with Dam methyltransferase according to the manufacturer's protocol and spin purified prior to transformation.

### Example 3

The use of nuclear selection to co-select for nuclear edits was used as a positive control for comparison against chloroplast selection. A nuclear targeting ribonucleoprotein (RNP) complex (T161-RNP) containing a nuclear guide RNA and recombinant Cas9 nuclease was tested for nuclear editing by co-transformation with either the circular chloroplast integration vector NAS30505, or the nuclear marker NAS16305-PCR (SEQ ID NO 10). The nuclear site targeted here, named T161 nuclear Cas9 target (SEQ ID NO 11), was chosen because it was shown to be a highly efficient nuclear target when utilizing nuclear markers, and thus was selected as a good test for nuclear editing capabilities utilizing a chloroplast marker.

For transformation, the two DNA constructs were separately precipitated onto gold nanoparticles alongside the T161-RNP complex and carried forward for biolistic transformation and selection on agar plates containing the appropriate selection agent—erythromycin for chloroplast selection and blasticidin for nuclear selection. Colonies of the Trebouxiophyte green alga Oocystis sp. were allowed to grow on the selection-agar plates for two weeks. Surviving colonies were screened individually to assess Cas9 activity at the nuclear T161 locus. PCR analysis was performed by amplifying across the T161 nuclear Cas9 target site (SEQ ID NO 11) using the forward primer T161_F_SCRN (SEQ ID NO 12) and reverse primer T161_R_SCRN (SEQ ID NO 13). The PCR reaction generated a DNA product 616 base-pairs in length when the locus is wild type. The PCR product was subsequently sequenced using the Sanger method and analyzed through a DNA-visualization software. Small insertions or deletions (aka “indels”) causing changes in the sequence at the targets site relative to the wildtype sequence were considered evidence of Cas9 activity followed by imperfect repair of the Cas9 induced double stranded break via non-homologous end joining (NHEJ). Additionally, reactions where no amplicon was produced were considered knockouts for the purpose of this study. These were found to usually be the result of NHEJ mediated concatemer insertions of the transformed vector.

For one of the transformations (Tx1), a plastome-targeting RNP (called RS3-cpRNP) was co-precipitated onto the same particle alongside the nuclear targeting T161-RNP. The RS3-cpRNP contains guide RNA targeting the plastome at the RS3 integration site (RS3 plastome Cas9 target, SEQ ID NO: 14) and was shown to increase transformation efficiency and greatly speed up the process of achieving homoplasmy. Tests were done with and without this RS3-cpRNP to determine whether it interferes with the delivery or activity of the nuclear targeting T161-RNP.

### Example 4

A total of five transformations were performed and analyzed as described above. The results showed that strains having nuclear genome modifications can be generated and isolated by utilizing a chloroplast selection marker. Cas9 editing efficiencies were calculated by dividing the number of knockouts by the number of colonies screened. As shown in Table 1 the Cas9 editing efficiencies utilizing the chloroplast marker ranged from 4.16% to 8.33% while Cas9 editing efficiencies utilizing the nuclear marker ranged from 28.26% to 36.5%. The data show a clear correlation between a chloroplast marker being delivered to the cell, integrating into the chloroplast, and conferring survival on the antibiotic with a nuclear-target RNP being delivered to the cell and generating a nuclear genome modification.

Previous experiments showed that isolating a cell with Cas9 activity at the target site without employing any type of selection would be restrictively low (1/10,000-1/10,000,000). Table 1 presents Cas9 editing results using chloroplast selection compared to nuclear selection.

TABLE 1

| | | | | | # colonies | |
|---|---|---|---|---|---|---|
| | | | | | showing | |
| | | | | | Cas9 | |
| | | Nuclear | Plastome | | activity at | Cas9 |
| | Selectable | targeting | targeting | Colonies | nuclear | editing |
| Transformation | marker | RNP | RNP | screened | target | efficiency |
| Tx #1 | NAS30505 | T161 | RS3 | 48 | 1 | 2.08% |
| | vector | | | | | |
| | (chloroplast | | | | | |
| | marker) | | | | | |
| Tx #2 | NAS30505 | T161 | none | 48 | 4 | 8.33% |
| | vector | | | | | |
| | (chloroplast | | | | | |
| | marker) | | | | | |
| Tx #3 | NAS30505 | T161 | none | 48 | 2 | 4.16% |
| | vector | | | | | |
| | (chloroplast | | | | | |
| | marker) | | | | | |
| Tx #4 | NAS16305- | T161 | none | 46 | 13 | 28.26% |
| | PCR (nuclear | | | | | |
| | marker) | | | | | |
| Tx #5 | NAS16305 - | T161 | none | 41 | 15 | 36.5% |
| | PCR (nuclear | | | | | |
| | marker | | | | | |

Cas9 editing efficiencies on a nuclear target were higher for nuclear selection markers than for chloroplast selection markers. This may be due to a higher event-correlation between the nuclear-target RNP and the nuclear DNA marker since they must both reach the same organelle in the given transformation, as opposed to a different organelle in the case where the chloroplast marker is employed. Cas9 activity for the pooled set of transformants generated by the nuclear marker was 32.18% (Table 2, Tx #4 and Tx #5). This efficiency was 5.33-fold higher than the pooled set of transformants generated by the chloroplast marker (the duplicate set without the chloroplast targeting RS3-cpRNP, or Tx #2 and Tx #3), which had an average Cas9 editing efficiency of 6.25%. This difference in Cas9 editing efficiency was shown to be statistically significant through a comparison of two proportions z-test, which yielded a p-value <0.00001.

Table 1 presents a pooled dataset for replicate transformations. Comparison of pooled transformations by two proportions t-test (Null hypothesis=Cas9 activity efficiency at T161 is equal when using a Chloroplast DNA marker vs using a Nuclear DNA marker. Alternate hypothesis=Cas9 activity efficiency at T161 is not equal when using a Chloroplast DNA marker vs using a Nuclear DNA marker). The value of z is −4.5046, the value of p is <0.00001. The results are significant at p<0.05.

TABLE 2

| | | | | | # colonies | |
|---|---|---|---|---|---|---|
| | | | | | showing | |
| | | | | | Cas9 | |
| | Selection | Nuclear | Plastome | Resulting | activity at | Cas9 |
| | DNA | targeting | targeting | colonies | nuclear | activity |
| Transformation | marker | RNP | RNP | screened | target | efficiency |
| Chloroplast | NAS30505 | T161 | none | 96 | 6 | 6.25% |
| marker pool, | vector | | | | | |
| no RS3 RNP | (chloroplast | | | | | |
| (Tx #2, #3) | marker) | | | | | |
| Nuclear | PCR- | T161 | none | 87 | 28 | 32.18% |
| marker pool | NAS16305 | | | | | |
| (Tx #4, #5) | (Nuclear | | | | | |
| | marker) | | | | | |

The data therefore show that according to the present invention one can select for a genetic modification in a nuclear genome by utilizing a chloroplast selectable marker. Clear correlations were demonstrated between nuclear editing events and chloroplast selection events. The invention therefore provides for easy tracking of selectable markers in the plastome with desired nuclear gene edits in species such as green algae where few nuclear markers are available.

### Example 5

This example demonstrates the ability to utilize a recyclable vector expressing a codA negative selection marker gene translationally fused to a spectinomycin resistance gene aadA (which encodes adenylyltransferase). The codA gene of E. coli encodes cytosine deaminase, which hydrolizes 5-Fluro-cytosine (5-FC) into the toxic 5-fluoro-uracil (5-FU). When codA is expressed and the organism is grown in the presence of 5-FC the conversion into the toxic 5-FU creates negative pressure and selects for loss of the gene via recombination of flanking direct repeats.

In this instance the direct repeat was the last 200 bp from the 3′ end of the RS3 up arm, which was placed just upstream of the RS3 down arm and downstream of the negative selection cassette. The approach involved 1-2 rounds of streaking and isolation on agar plates containing 5-FC and resulted in total loop-out and loss of the DNA between the direct repeats. Since the direct repeats were native sequences in their native position, the resulting plastome reverted to wild-type and was indistinguishable from the native plastome. Long-read nanopore sequencing detected no residual transformed DNA in the nuclear, mitochondrial, or chloroplast genomes. This illustrated the convenient use of native direct repeats for scarless marker excision. When used in combination with chloroplast markers to enable nuclear editing, it enabled the user to avoid permanently using up nuclear markers while editing a nuclear genome. The data showed that no transformed DNA was left behind. This approach is particularly useful in applications where non-GMO organisms are desirable.

Although the invention has been described with reference to the presently preferred embodiment, it should be understood that various modifications can be made without departing from the spirit of the invention. Accordingly, the invention is limited only by the following claims.

What is claimed is:

1. A method of selecting for a genetic modification to a genome in the nucleus of a photosynthetic cell comprising:
transforming the photosynthetic cell with a ribonucleoprotein complex that effects a genetic modification in the genome in the nucleus of the photosoynthetic cell;
transforming the photosynthetic cell with a construct that effects an insertion of a selectable marker into the plastid of the photosynthetic cell;
selecting for photosynthetic cells comprising the selectable marker inserted into the plastid;
thereby selecting for the genetic modification to the genome in the nucleus of the photosynthetic cell.

2. The method of claim 1, wherein the insertion of the selectable marker into the plastid of the cell comprises inserting the selectable marker into the plastome.

3. The method of claim 1, wherein the plastid is a chloroplast.

4. The method of claim 1, further comprising sequencing at least a portion of the genome in the nucleus of the photosynthetic cell to confirm the genetic modification has been performed.

5. The method of claim 1, wherein selecting for photosynthetic cells comprises plating algal cells and selecting colonies comprising the insertion into the plastome.

6. The method of claim 1, wherein the selectable marker inserted into the plastome enables the photosynthetic cell to grow on an antibiotic.

7. The method of claim 1, wherein the antibiotic is erythromycin or spectinomycin.

8. The method of claim 1, wherein the selectable marker is glufosinate ammonium or the ptxD gene.

9. The method of claim 1, wherein the cells are plated onto selection agar plates.

10. The method of claim 1, wherein the ribonucleoprotein complex comprises a guide RNA targeted to a sequence on the genome in the nucleus of the photosynthetic cell and a Cas nuclease.

11. The method of claim 1, wherein the method does not comprise screening for a selectable marker comprised in the genome of the nucleus of the photosynthetic cell.

12. The method of claim 1, wherein the photosynthetic cell is an algal cell, and the algal cell is a Trebouxiophyte algal organism.

13. The method of claim 12, wherein the Trebouxiophyte algal organism is from the genus Oocystis.

14. The method of claim 1, wherein the construct that effects an insertion of a selectable marker into the plastid comprises RS-up and RS-down arms that recombine with homologous sequences in the plastome to thereby effect insertion; and the construct further comprises a selectable marker cassette in between the RS-up and RS-down arms.

15. The method of claim 1, any one of claims 1-14 wherein the construct is amplified by PCR prior to being transformed into the plastid.

16. The method of claim 1, wherein the construct further comprises a chloroplast promoter and chloroplast terminator that controls expression of the encoded selectable marker.

17. The method of claim 16, wherein the encoded selectable marker confers the ability to grow on media containing erythromycin, spectinomycin, or glufosinate ammonium.

18. The method of claim 16 wherein the encoded selectable marker comprises the ptxD gene.

19. The method of claim 1, wherein the transformations are performed using biolistics.

20. A composition comprising a ribonucleoprotein complex for editing a nuclear genome, and a construct comprising a selectable marker gene comprised in between two restriction site arms, each restriction site arm being homologous to corresponding sequences on a plastid genome.

21. The composition of claim 20, wherein the ribonucleoprotein complex comprises a guide RNA targeted to a site on the nuclear genome of a subject organism.

22. The composition of claim 20, wherein the ribonucleoprotein complex comprises Cas9.

23. The composition of claim 21, wherein the subject organism is a Chlorophyte organism.

24. The composition of claim 23 wherein the Chlorophyte organism is a Trebouxiophyte alga.

25. The composition of claim 20, adsorbed to a microcarrier for biolistics.

26. A kit comprising the composition claim 20.
